(19) United States
(12) Patent Application Publication
(10) Pub. No.: US 2007/0104811 A1
(43) Pub. Date: May 10, 2007

(54) Pharmaceutical composition useful for the treatment of prostate cancer

(75) Inventors: Swati Sinha, Kolkata (IN); Bikas Chandra Pal, Kolkata (IN); Samir Bhattacharya, Santineketan (IN); Deepankar Dutta Gupta, Kolkata (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/511,874

(22) Filed: Aug. 29, 2006

(30) Foreign Application Priority Data
Sep. 1, 2005 (IN) 2335/DEL/2005

(51) Int. Cl. A61K 36/185

(52) U.S. Cl. 424/774

(57) ABSTRACT

Present invention provides a pharmaceutical composition useful for the treatment of prostate cancer in a subject wherein the said composition comprising the therapeutically effective amount of an extract obtained from any plant parts of Murrya koenigii and Tribulus terrestris.

### FIELD OF INVENTION

The present invention relates to a pharmaceutical composition useful for the treatment of prostate cancer in a subject. Further, it relates to a method of treating prostrate cancer in a subject.

The present invention also relates to a process for the preparation of a pharmaceutical composition comprising an extract obtained from the leaves or any other plant part of Murraya koenigii and Tribulus terrestris useful for treatment of prostate cancer.

### BACKGROUND AND PRIOR ART OF INVENTION

One of the prevalent forms of neoplasia afflicting men above the age of 65 years is prostate cancer. Mortality from Prostate cancer results from metastasis to bones and lymph nodes. Early detection through serum testing of Prostate Specific Antigen (PSA), improved surgical intervention, radiation therapy, androgen ablation have yielded no answer to the patients who suffer from recurrent or residual cancer (Chinni S R, Li Y, et. al. Indole 3 carbinol(I3C) induced cell growth inhibition, G1 cell cycle arrest and Apoptosis in prostate cancer cells. Oncogene;20:2927-2936(2001). Popular, current therapy of Androgen ablation almost invariably leads to aberrant expression and interaction of Tyrosine kinase and associated ligands. Several recent studies have shown constitutively active mitogenic and cell survival signaling in Prostate cancer. The delicate link between cellular proliferation and apoptosis is challenged by such cellular aberrations. The primary mechanism i.e. prostate carcinoma by-pass apoptosis is by the up regulation of PI3Kinase/Akt survival pathway (Li Y, Sarkar F H. Inhibition of nuclear factor κB activation in PC-3 cells by genistin is mediated via Akt signaling pathway. Clin. Cancer. Res;8: 2369-2377(2002). Akt related serine-threonine in signaling cascade that regulates cell survival are important in the pathogenesis of cancer (Chinni S R, Sarkar F H. Akt inactivation is a key event in Inole 3 carbinol induced apoptosis in PC-3 cells. Clin. Cancer Res; 8: 1228-1236(2002). It inactivates a range of pro apoptotic proteins like Bad, forkhead transcription factor, caspase 9 (Green D R, Reed J C. Mitochondria and Apoptosis. Science; 281: 1309-1312(1998); Thomberry N A, Lazebnik Y. Caspases: enemies within. Science; 281: 1312-1316(1998) while activating Bcl-2 (Adams J M, Cory S. The Bcl-2 protein family Arbiters of cell survival. Science; 281: 1322-1326(1998), NFγB like anti-apoptotic proteins (Datta S R, Brunet A, Greenberg M E. Cellular survival: A play in three Akts. Genes and Dev; 13: 2905-2927(1999).

### OBJECTS OF THE INVENTION

The main object of the present invention is to provide a pharmaceutical composition useful for the treatment of prostate cancer in a subject.

Further another object of the present invention is to provide a method of treating prostrate cancer in a subject.

Yet another object of the present invention is to provide a process for the preparation of said pharmaceutical composition comprising an extract obtained from the leaves or any other plant part of Murraya koenigii and Tribulus terrestris useful for treatment of prostate cancer wherein the said process comprises of homogenizing the dried leaves or any other plant part of Murraya koenigii and Tribulus terrestris with water wherein the ratio used is 1:1 followed by freeze dried.

Still another objective of the present invention is to determine cell death, release of cytochrome c, activation caspase cascade, cleavage of PARP DNA repair enzyme, along with the down regulation of Bc1-x1 and pAkt, with the extract obtained from Murraya koenigii and Tribulus terrestris.

### SUMMARY OF THE INVENTION

Present invention provides a pharmaceutical composition useful for the treatment of prostate cancer in a subject wherein the said composition comprising the therapeutically effective amount of an extract obtained from any plant parts of Murrya koenigii and Tribulus terrestris. Further, it also deals with a process for the preparation of extract from the leaf extract or any other plant parts of Murraya koenigii, which efficiently kills androgen dependent and independent Prostate cancer cells.

### BRIEF DESCRIPTION OF FIGURES

FIG. 1 represents effect of separate leaf extract on PC-3 cell line as well as dose response to PC-3 cell line to combined leaf extract of M. koenigii and T. terrestris. (200 μg/ml) and T. Terrestris (200 μg/ml) sepeartely and M. Koenigii+T. Terrestris (100 μg/ml+100 μg/ml) combined in comparison to untreated cells(control) by MTT assay.

FIG. 2 represents cell viability assessment with M. koenigii and T. terrestris. Combined extracts did not show any toxic effects on neonatal skeletal muscle cell, cardiomyocyte, hepatocyte with the same dose i.e 100 μg/ml M. koenigii+100 μpg/ml T. terrestris total amounting to of 200 μg/ml suggesting the specificity of the combined extracts for inducing apoptosis in prostate cancer cells.

FIG. 3 represents inhibition of Akt phosphorylation by M. koenigii and T. terrestris. The bi-herbal extract inhibited phosphorylation of Akt in a dose and time dependent manner. It showed no effect on the steady state levels of Akt protein but its serine 473 phosphorylation was inhibited at 24 h and by 36 h its activation was blocked.

FIG. 4 represents inhibition of Bcl-x1 expression by M. koenigii and T. terrestris. Bcl-x1 is a mitochondrial membrane protein that maintains mitochondrial membrane integrity in survival signal pathway.

FIG. 5 represents caspase 3 activation by M. koenigii and T. terrestris indicating initiation of apoptotic pathway. Down regulation of Bcl-x1 permits disintegration of mitochondrial outer membrane that causes leakage of cytochrome c initiating the caspase cascade. The activation of caspase 3 was detected.

### DETAILED DESCRIPTION OF THE INVENTION

Accordingly, the present invention provides a pharmaceutical composition useful for the treatment of prostate cancer in a subject wherein the said composition comprising the therapeutically effective amount of an extract obtained from any plant parts of Murrya koenigii and Tribulus terrestris.

Both leaf extracted fraction from M. koenigii shows considerable activity in the induction of apoptosis in two androgen independent Prostate cancer cell lines PC-3 and androgen dependent Prostate cancer cell line LNCaP.

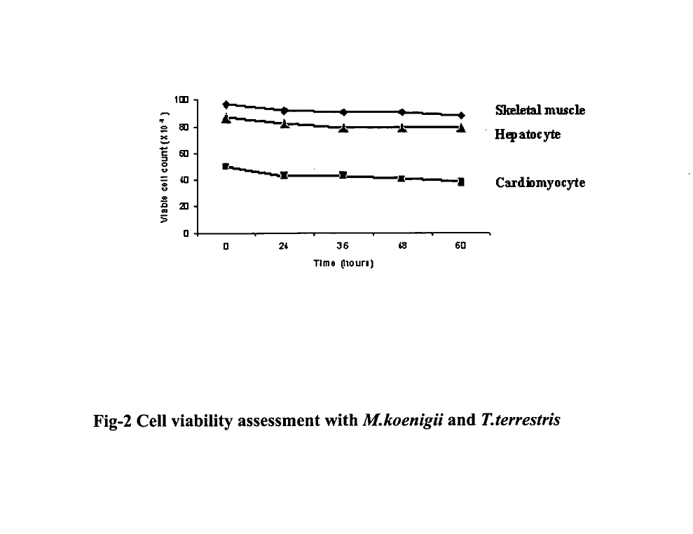

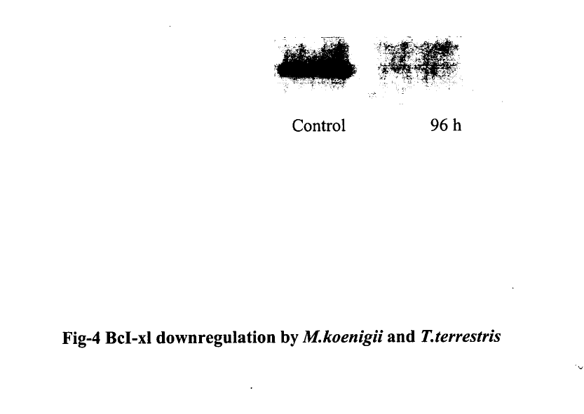

The leaf extract from M. koenigii induced apoptosis of androgn independent and androgen dependent prostate cancer cells in a time and dose dependent manner. Leaf extract caused significant apoptosis of all these cell lines (75% cell death in 96 h) with 200 μg/ml dosage. When the same treatment was performed with Tribulus terrestris extracts, it affected cell viability of above mentioned cell line but it had much weaker effect (more than 50% cell death at 96 h) with the same dose (200 μg/ml of culture media). However, a combination of M. koenigii and T. terrestris extracts showed increased levels cell death in comparision to M. koenigii extract suggesting a synergistic action of T. terrestris extract when combined with M. koenigii. The combined leaf extract from and Tribulus terrestris and M. koenigii induced apoptosis of androgen independent and androgen dependent prostate cancer cells in a time and dose dependent manner. Combined extracts caused significant apoptosis of all these cell lines at a dose of 100 μg/ml M koenigii+100 μg/ml T. terrestris total amounting to of 200 μg/ml at 96 h (about 95% cell death). FIG. 2 shows the data of PC-3 cell line. Effect of the extract and purified compound on other cells i.e neonatal skeletal muscle cell, cardiomyocyte, hepatocyte have been examined as control cells. At the above mentioned dose and time there was no sign of cell death( FIG. 4). It also induced the caspase cascade and down regulation of pAkt as well as Bcl-x1 expression, the most important survival signals in cancer cells.

In an embodiment of the present invention, the said composition comprises the therapeutically effective amount of an extract obtained from any plant parts of Murrya koenigii and Tribulus terrestris optionally along with one or more pharmaceutically acceptable carriers.

In another embodiment of the present invention, the dosage of the said composition is administered at a unit dose of at least 10-15 mg/kg body weight.

In still another embodiment of the present invention, the dosage of the said composition is administered at a unit dose of at least 0.1-5 mg/kg body weight.

In still another embodiment of the present invention, the dosage of the said composition is administered preferably in water-soluble form.

In still another embodiment of the present invention, the said carriers are selected in such a manner that it does not interfere with the activity of fraction of Murrya koenigii extract.

In still another embodiment of the present invention, wherein the said carriers is selected from the group consisting of proteins, carbohydrates, sugar, talc, magnesium stearate, cellulose, calcium carbonate, and starch-gelatin paste and pharmaceutical acceptable carriers, excipient, diluent or solvent.

In still another embodiment of the present invention, the administration route is selected from the group consisting of oral, intravenous, intramuscular or subcutaneous route.

In still another embodiment of the present invention, the said form for oral route is selected from the group consisting of capsule, syrup, concentrate, powder and granules. Further, the present invention provides a method of treating prostrate cancer in a subject, wherein the said method comprising the step of administering to the subject a pharmaceutical composition comprising the therapeutically effective amount of an extract obtained from any plant parts of Murrya koenigii and Tribulus terrestris. In an embodiment of the present invention, the said method comprising the step of administering to the subject a pharmaceutical composition comprises the therapeutically effective amount of an extract obtained from any plant parts of Murrya koenigii and Tribulus terrestris optionally along with one or more pharmaceutically acceptable carriers.

In another embodiment of the present invention, the dosage of the above said composition is administered at a unit dose of at least 10 g-15 mg/kg body weight.

Further, in an embodiment of the present invention, the said method comprising the step of administering to the subject a pharmaceutical composition.

In still another embodiment of the present invention, the dosage of the above said formulation is administered at a unit dose of at least 0.1-5.0 mg/kg body weight.

In still another embodiment of the present invention, the dosage of the said composition is administered preferably in water-soluble form.

In still another embodiment of the present invention, the said carriers is selected from the group consisting of proteins, carbohydrates, sugar, talc, magnesium stearate, cellulose, calcium carbonate, and starch-gelatin paste and pharmaceutical acceptable carriers, excipient, diluent or solvent.

In still another embodiment of the present invention, the administration route is selected from the group consisting of, oral, intravenous, intramuscular or subcutaneous route.

In still another embodiment of the present invention, the said form for oral route is selected from the group consisting of capsule, syrup, concentrate, powder and granules.

In still another embodiment of the present invention, the androgen independent cell line PC 3 and androgen dependent cell line LNCaP is killed by the said composition in a dose and time dependent manner.

In still another embodiment of the present invention, the phosphorylation of Akt is inhibited by the said composition in a dose and time dependent manner.

In still another embodiment of the present invention, other cells such as hepatocytes, cardiomayocytes, and skeletal muscle are not killed by the said composition.

The present invention also provides a process for the preparation of an pharmaceutical compostion comprising an extract obtained from the leaves or any other plant part of Murraya koenigii and Tribulus terrestris useful for treatment of prostate cancer wherein the said process comprises of homogenizing the dried leaves or any other plant part of Murraya koenigii and Tribulus terrestris with water wherein the ratio used is 1:1 followed by freeze dried.

In an embodiment of the present invention, the plant parts are selected from the group consisting of leaves, stems, fruits, or any other part of the Murrya Koenigii and Tribulus terrestris.

In another embodiment of the present invention, the leaves of Murraya koenigii and Tribulus terrestris is collected from different areas of West Bengal, India.

In further another embodiment of the present invention, the leaves used are taken from fresh or/and sun shade dried leaves of Murrya Koenigii and Tribulus terrestris.

Further, in another embodiment of the present invention, the said composition is prepared by mixing the extract obtained from Murraya koenigii and Tribulus terrestris in the ratio 1:1.

In still another embodiment of the present invention, the anti-carcinogenic activity of the said extract is confirmed by in-vivo experiments.

In still another embodiment of the present invention, the use of the said extract is in the treatment of prostate cancer.

In another embodiment of the present invention, the anti-carcinogenic activity of the said compound is confirmed by in-vivo experiments.

The following examples are given by way of illustration of the present invention and should not be construed to limit the scope of present invention.

Example 1 to 8 relates the extract obtained from Murraya koenigii (Rutaceae) and Tribulus terrestris and its activity thereof.

### EXAMPLE 1

Sourse of Murraya koenigii (Rutaceae) and Tribulus terrestris

The leaves of Murraya koenigii (Rutaceae) and Tribulus terrestris were collected from different areas of West Bengal, India. A voucher specimen has been deposited at the Department of Medicinal Chemistry, Indian Institute of Chemical Biology, Kolkata, India.

### EXAMPLE 2

Isolation of Extract of Murraya koenigii (Rutaceae) and Tribulus terrestris

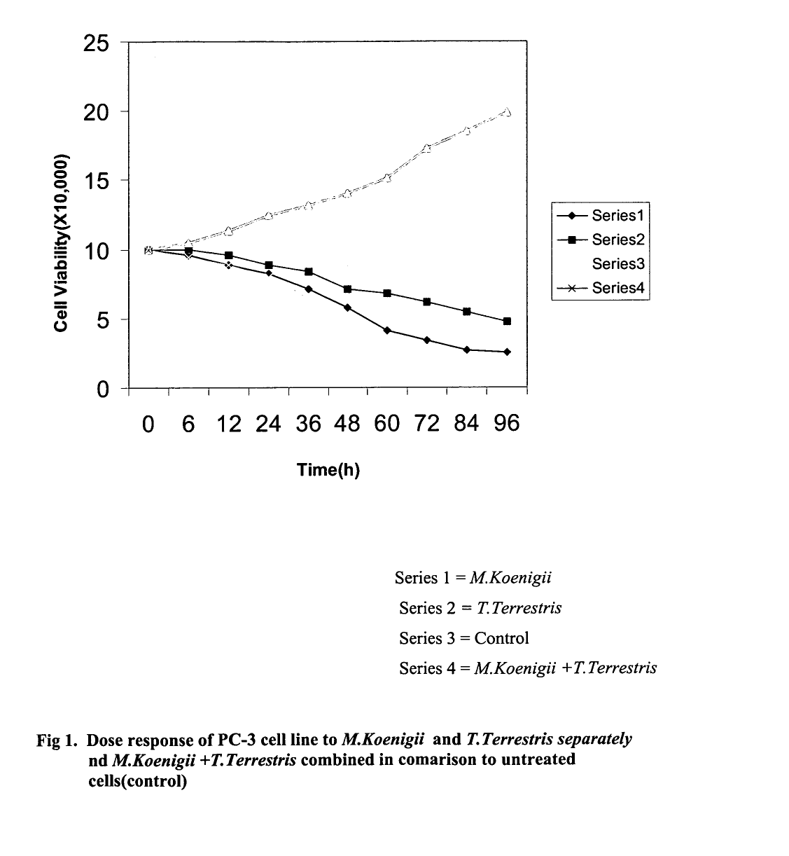

The fresh leaves and all other plant parts of Murraya koenigii (1.2 Kg) was homogenized with water (1.5 lit) in a mixture-blender and freeze dried. Activity of the freeze-dried material was examined on prostate cancer cell line. The detail of cell death by the material has been described under the FIG. 1. Dry powder of Tribulus terrestris (0.5 kg) was procured in similar manner and tested for biological activity. Combined extracts caused significant apoptosis of all these cell lines at a dose of 100 μg/ml M. koenigii+100 μg/ml T. terrestris total amounting to of 200 μg/ml of culture media at 96 h (about 95% cell death).

### EXAMPLE 3

Culturing of Human Prostate Cancer Cell Line, PC-3

Human prostate cancer cell line, PC-3 (PTEN -ve, androgen independent) from American Type Culture Collection, Manassas, Va., USA. Cells were grown in Dulbecco's Modified Eagle Medium supplemented with 10% fetal bovine serum and 1% antibiotic-antimycotic. Cells were cultured in 37° C. in an atmosphere of 5% CO₂.

Primary Culture of Rat Neonatal Cardiomyocytes

The cardiomyocytes from 2-day-old neonatal rat were isolated by the method previously described by Yoshihiro Kimura (1994) with modifications. Briefly, heart was excised and minced in pre warmed (37° C.) Ads buffer (1.2M NaCl, 198 mM HEPES, 54 mM KCl, 8.3 mM MgSO₄, 55.4 mM glucose, 95 mM NaH₂PO₄), digested in typeII Collagenase 0.05% and Pancreatin in 3-succesive digestions of 15 minutes each. Supernatant was pooled and cells palleted at 2000 g, 10 minutes. Cells were resuspended and plated in collagen coated T-25 flaks in Medium-199 enriched with 10% Fetal Bovine Serum and 1% antibiotic-antimycotic.

Primary Culture of Rat Neonatal Hepatocyte

The hepatocytes from 2-day-old neonatal rat were isolated by method described previously by William E Russell (1997) with modifications. Briefly, the livers were perfused through the portal vein with a calcium-free solution consisting of 150 mM NaCl, 2.8 mM KCl, 5.5 mM glucose, and 25 mM HEPES (pH 7.6) for 10 min, followed by mincing and digestion in DMEM containing 0.05% collagenase type IV for 30 minutes. The cells were centrifuged (2000 g, 10 minutes) and dispersed in DMEM enriched with 10% fetal Bovine Serum, 1% antibiotic-antimycotic.

Primary Culture of Neonatal Rat Skeletal Muscle

The skeletal muscles from 2-day-old neonatal rat were isolated by method described previously by William E Russell (1997) with modifications. Briefly, Soleus muscle were dissected, minced and digested with 0.2% Type II Collagenase and 0.05% trypsin in Phosphate buffered Saline (PBS) pH 7.4, 0.15M NaCl. The dispersed skeletal muscle cells were centrifuged (1000 g, 10 minutes) washed and resuspended in Phosphate buffered Saline (PBS) with 1% antibiotic-antimycotic. Cells were preincubated for 30 minutes at 37° C., 95% air/5%CO2. The floating muscle cells were plated on collagen coated T-25 flaks in Dulbecco's modified Eagle Medium enriched with 10% Fetal. Bovine Serum and 1% antibiotic-antimycotic

### EXAMPLE 4

Viability of PC-3 after treatment as at the indicated times was determined by MTT assay -Cell titre 96 AQoueus One solution Cell Proliferation Assay (Promega, Corp. Madison, Wis.) as per manufacturer's protocol. Briefly, 10,000 cells were plated in triplicate in 96 well plates and incubated for 12 hours in complete media. Compound MK-3 was added as indicated on replacing them to fresh complete media and incubated upto 96 hours for varied time periods. 20 μl per well of cell titre 96 AQoueus One solution reagent. Incubate the plate for 1-4 hours at 37° C. and humidified 5% CO₂ atmosphere. Absorbance was recorded at 490 nm with 96 well plate readers. Reference wavelength of 630 nm was used to reduce background contributed by non-specific absorbance due to cell debris.

### EXAMPLE 5

Electrophoresis and Immunoblotting

PC-3 cells (1×10⁶ cells) were incubated with complete medium. Cells were detached using cell dissociation reagent from Sigma Chemical company St Louis, Mo., USA. They were collected by centrifugation at 1500 g for 10 mins and boiled for 5-7 minutes in sodium dodecyl sulphate(SDS) buffer (pH 6.8). Aliquots containing 60 μg total cellular protein were separated by 10% SDS-PAGE and transferred to PVDF membrane (MILIPORE, Bedford, Mass., USA). Membrane was blocked with blocking buffer for 1 hour at room temperature and probed with desired primary antibody caspase-9, Bcl-x1, pAktl/2/3(ser 473), anti PARP, Cytochrome C (Santa-Cruz Biotechnology, Inc. USA). Anti Caspase-3 that recognizes procaspse-3 (32 kDa) and the active cleaved caspase-3 (17 kDa) (BD Biosciences, Mountainview, Calif.). Akt, cleaved caspase-9 (Cell Signaling technology, Beverly, Mass.) overnight at 4° C. followed by alkaline phosphatase conjugated secondary antibody and detection.

### EXAMPLE 6

To search anti-carcinogenic activity from medicinal plants of India, we selected Murraya koenigii and Tribulus terrestris leaves extract and observed efficient killing of androgen independent prostate cancer cell line PC-3 (FIG. 1). It shows the data of PC-3 cell line.

Combined extract of M. koenigii and T. terrestris reduces PC-3 cell viability by inducing Apoptosis Combined extracts did not show any toxic effects on neonatal skeletal muscle cell, cardiomyocyte , hepatocyte with the same dose i.e 100 μg/ml M. koenigii+100 μg/ml T. terrestris total amounting to of 200 μg/ml of culture media (FIG. 2) suggesting the specificity of the combined extracts for inducing apoptosis in prostate cancer cells.

In-Vivo Experiment to Confirm In-Vitro Results

Experiments for acute toxicity are performed as per the guidelines of WHO. Experimental subject: Mice, Balb/C Male, female. 25 gms body weight (approx) 16 mice were divided into 2 groups 8 animals were received the dosage of @2 gm/kg body weight of combined extract. Equal volume of the vehicle (DMSO) were given to 8 animals wherein dose administered orally once and then animals sacrificed and haematological parameters checked 15 days after treatment.

TABLE 1
Haematological tests for crude extract:

| | RBC | WBC | Hb(gm/dl) |
|---|---|---|---|
| Control | 1029 | 92 | 10.69 |
| Treated | 1032 | 89 | 10.713 |

There was no indication of any toxicity in treated mice (table 1)

### EXAMPLE 7

Inhibition of Cell-Survival Pathway Bi-Herbal Extract Obtained from M. koenigii and T. terrestris Treated PC-3 Cells

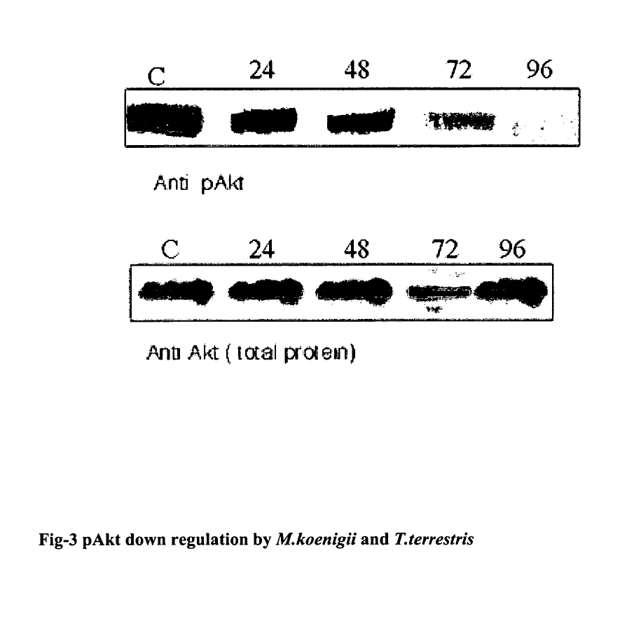

In cell survival pathway, Akt kinase plays an important role by inhibiting apoptotic processes. Akt phosphorylates downstream effector molecules such as pro-apoptotic protein Bad effecting its inactivation. This does not permit its dimerisation with Bcl-x1 that result in the inhibition of apoptotic process. The bi-herbal extract inhibited phosphorylation of Akt in a dose and time dependent manner. It showed no effect on the steady state levels of Akt protein but its serine 473 phosphorylation was inhibited at 24 h and by 36 h its activation was totally blocked (FIG. 3). The basal level of phosphorylation at serine 473 is predominant in PC-3 cell line. The inactivation of Akt phosphorylation could be attributed to the inactivation of cell survival pathways resulting subsequent induction of apoptosis in treated PC-3 cells.

It has been then investigated whether mahanine bi-herbal extract obtained from M. koenigii and T. terrestris) induced apoptosis of PC-3 cells is effected through Bcl-x1. Bcl-x1 is a mitochondrial membrane protein that maintains mitochondrial membrane integrity in survival signal pathway. The bi-herbal extract significantly decreased Bcl-x1 expression at 36 h (FIG. 4).

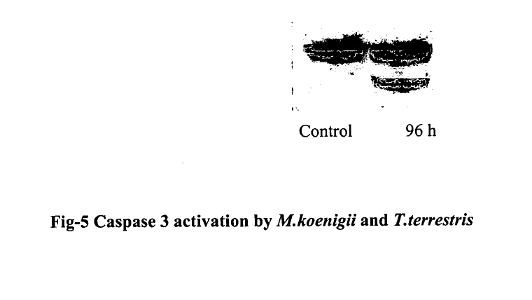

This indicates initiation of apoptotic pathway as down regulation of Bcl-x1 permits disintegration of mitochondrial outer membrane that causes leakage of cytochrome c initiating the caspase cascade. The activation of caspase 3 was detected at 48h and peak of the activity could be detected at 60 h (FIG. 5).

### EXAMPLE 8

Therapeutic Evaluation of the preparation developed by combination of M. koenigii leaves and T. terrestris seed extract for the treatment of prostate cancer (Conducted by registered Auyrvedic practioner). 3 capsules per day (each containing 330 mg) were administered orally.

| | | weight of prostate |
|---|---|---|
| | weight of prostate | after 2 months |
| Patient | before treatment | of treatment |
| A | 39 gm | 21 gm |
| B | 46 gm | 25 gm |
| C | 34 gm | 18 gm |
| D | 42 gm | 27 gm |
| E | 29 gm | 16 gm |
| F | 41 gm | 19 gm |

All the above patients reported easy flow of urine during both day and night after taking capsules for 2 months

Example 9 to 15 relates the compound mahanine obtained from the extract of Murraya koenigii and its activity thereof.

### EXAMPLE 9

Human prostate cancer cell line, PC-3 (PTEN-ve, androgen independent) from American Type Culture Collection, Manassas, Va., USA. Cells were grown in Dulbecco's Modified Eagle Medium supplemented with 10% fetal bovine serum and 1% antibiotic-antimycotic. Cells were cultured in 37° C. in an atmosphere of 5% CO₂.

Primary Culture of Rat Neonatal Cardiomyocytes

The cardiomyocytes from 2-day-old neonatal rat were isolated by the method previously described by Yoshihiro Kimura (1994) with modifications. Briefly, heart was excised and minced in pre warmed (37 degree C.) Ads buffer (1.2M NaCl, 198 mM HEPES, 54 mM KCl, 8.3 mM MgSO₄, 55.4 mM glucose, 95 mM NaH₂PO₄), digested in typeII Collagenase 0.05% and Pancreatin in 3-succesive digestions of 15 minutes each. Supernatant was pooled and cells palleted at 2000 g, 10 minutes. Cells were resuspended and plated in collagen coated T-25 flaks in Medium-199 enriched with 10% Fetal Bovine Serum and 1% antibiotic-antimycotic.

Primary Culture of Rat Neonatal Hepatocyte

The hepatocytes from 2-day-old neonatal rat were isolated by method described previously by William E Russell (1997) with modifications. Briefly, The livers were perfused through the portal vein with a calcium-free solution consisting of 150 mM NaCl, 2.8 mM KCl, 5.5 mM glucose, and 25 mM HEPES (pH 7.6) for 10 min, followed by mincing and digestion in DMEM containing 0.05% collagenase type IV for 30 minutes. The cells were centrifuged (2000 g, 10 minutes) and dispersed in DMEM enriched with 10% fetal Bovine Serum, 1% antibiotic-antimycotic.

Primary Culture of Neonatal Rat Skeletal Muscle

The skeletal muscles from 2-day-old neonatal rat were isolated by method described previously by William E Russell (1997) with modifications. Briefly, Soleus muscle were dissected, minced and digested with 0.2% Type II Collagenase and 0.05% trypsin in Phosphate buffered Saline (PBS) pH 7.4, 0.15M NaCl. The dispersed skeletal muscle cells were centrifuged (100 g, 10 minutes) washed and resuspended in Phosphate buffered Saline (PBS) with 1% antibiotic-antimycotic. Cells were preincubated for 30 minutes at 37° C., 95% air/5% CO2. The floating muscle cells were plated on collagen coated T-25 flaks in Dulbecco's modified Eagle Medium enriched with 10% Fetal Bovine Serum and 1% antibiotic-antimycotic.

### EXAMPLE 10

Viability of PC-3 after treatment as at the indicated times was determined by MTT assay-Cell titre 96 AQoueus One solution Cell Proliferation Assay (Promega, Corp. Madison, Wis.) as per manufacturer's protocol. Briefly, 10,000 cells were plated in triplicate in 96 well plates and incubated for 12 hours in complete media. Compound MK-3 was added as indicated on replacing them to fresh complete media and incubated upto 72 hours for varied time periods. 20 μl per well of cell titre 96 AQoueus One solution reagent. Incubate the plate for 1-4 hours at 37° C. and humidified 5% CO₂ atmosphere. Absorbance was recorded at 490 nm with 96 well plate readers. Reference wavelength of 630 nm was used to reduce background contributed by non-specific absorbance due to cell debris.

### EXAMPLE 11

Electrophoresis and Immunoblotting

PC-3 cells (1×10⁶ cells) were incubated with complete medium alone or with 3 μg/ml of Mahanine (MK-3) as indicated. Cells were detached using cell dissociation reagent from Sigma Chemical company St Louis, Mo., USA. They were collected by centrifugation at 1500 g for 10 mins and boiled for 5-7 minutes in sodium dodecyl sulphate(SDS) buffer (pH 6.8). Aliquots containing 60 μg total cellular protein were separated by 10% SDS-PAGE and transferred to PVDF membrane (MILIPORE, Bedford, Mass., USA). Membrane was blocked with blocking buffer for 1 hour at room temperature and probed with desired primary antibody caspase-9, Bcl-x1 , pAkt1/2/3(ser 473), anti PARP, Cytochrome C (Santa-Cruz Biotechnology, Inc. USA). Anti Caspase-3 that recognizes procaspse-3 (32 kDa) and the active cleaved caspase-3 (17 kDa) (BD Biosciences, Mountainview, Calif.). Akt, cleaved caspase-9 (Cell Signaling technology, Beverly, Mass.) overnight at 4° C. followed by alkaline phosphatase conjugated secondary antibody and detection.

### EXAMPLE 12

Immunofluorescence

Cells were cultured on glass cover slips. Both control and treated cells were fixed with PBS containing 4% paraformaldehyde for 2 h at 4 C. The cells were permiabilized with 0.1% Triton X-100 in PBS and then incubated with rabbit polyclonal anti-cytochrome c antibody (Santa cruz, USA, dil 1:50) for 3 h followed by incubation with FITC-conjugated secondary antibody (goat anti rabbit, Santa Cruz, 1:50) for another 1 h with rigorous washing in all the above steps with PBS. 1 μg/ml DAPI was also added in each set. The stained cells were observed under fluorescence microscope (Olympus BX51 microscope, Tokyo, Japan) and the images were captured with cool Snap Pro camera.

In-Vivo Experiment to Confirm In-Vitro Results

Experiments for acute toxicity are performed as per the guidelines of WHO. Experimental subject: Mice, Balb/C Male, female. 25 gms body weight (approx) 16 mice were divided into 2 groups 8 animals were received the compound @2 gm/kg body weight. Equal volume of the vehicle (DMSO) were given to 8 animals wherein dose administered orally once and then animals sacrificed and haematological and biochemical parameters checked 15 days after treatment.

TABLE 2(a)
Haematological tests for Compound mahanine:

| | RBC | WBC | Hb(gm/dl) |
|---|---|---|---|
| Control | 1029 | 92 | 10.69 |
| Treated | 1032 | 89 | 10.713 |

Advantages:

The main advantages of the present invention are:
1. Present invention provides a simplified method of bioactive extraction and a simplified fast and inexpensive process for the preparation of fraction mahanine possessing significantly high biological activities relevant to treatment, relief and remedy of prostate cancer.
2. It also provides a pharmaceutical composition which is highly compatible for human consumption and capable for being used for the treatment, relief and remedy of prostate cancer.

1. A pharmaceutical composition useful for the treatment of prostate cancer in a subject wherein the said composition comprising the therapeutically effective amount of an extract obtained from any plant parts of Murrya koenigii and Tribulus terrestris optionally along with one or more pharmaceutically acceptable carriers.

2. The pharmaceutical composition as claimed in claim 1, wherein the extract obtained from any plant parts of Murrya koenigii and Tribulus terrestris used is in the ratio of about 1:1.

3. A method as claimed in claim 9, wherein the dosage of the said composition administered is at a unit dose of at least 10 mg-15 mg /kg body weight.

4. A pharmaceutical composition as claimed in claim 1, wherein the dosage of the said composition is administered preferably in water-soluble form.

5. A pharmaceutical composition as claimed in claim 1, wherein the carrier used is selected from the group consisting of proteins, carbohydrates, sugar, magnesium stearate, cellulose, calcium carbonate and starch-gelatin paste.

6. A pharmaceutical composition as claimed in claim 1, wherein the administration route is selected from the group comprising of oral, intravenous, intramuscular or subcutaneous route.

7. A pharmaceutical composition as claimed in claim 6, wherein the said form for oral route is selected from the group consisting of capsule, syrup, concentrate, powder and granules.

8. A method of treating prostrate cancer in a subject, wherein the said method comprising the step of administering to the subject a pharmaceutical composition comprising the therapeutically effective amount of an extract obtained from any plant parts of Murrya koenigii and Tribulus terrestris with one or more pharmaceutically acceptable carriers.

9. A method as claimed in claim 8, wherein the said method comprising the step of administering to the subject a pharmaceutical composition useful for the treatment of prostate cancer in a subject wherein the said composition comprising the therapeutically effective amount of an extract obtained from any plant parts of Murrya koenigii and Tribulus terrestris optionally along with one or more pharmaceutically acceptable carriers.

10. A method as claimed in claim 9, wherein the dosage of the said composition administered is at a unit dose of at least 10 mg-15 mg/kg body weight.

11. A method as claimed in claim 10, wherein the androgen independent cell line PC 3 and androgen dependent cell line LNCaP is killed by the said composition in a dose and time dependent manner.

12. A method as claimed in claim 11, wherein the phosphorylation of Akt is inhibited by the said composition in a dose and time dependent manner.

13. A process for the preparation of an pharmaceutical composition comprising an extract obtained from the leaves or any other plant part of Murraya koenigii and Tribulus terrestris useful for treatment of prostate cancer wherein the said process comprises of homogenizing the dried leaves or any other plant part of Murraya koenigii and Tribulus terrestris with water followed by freeze dried wherein the ratio of the plant parts used is about 1:1.

14. A process as claimed in claim 13, wherein the plant parts are selected from the group consisting of leaves, stems, fruits, or any other part of the Murrya Koenigii and Tribulus terrestris.

15. A process as claimed in claim 14, wherein the leaves of Murraya koenigii and Tribulus terrestris is collected from different areas of West Bengal, India.

16. A process as claimed in claim 13, wherein the said composition is prepared by mixing the extract obtained from Murraya koenigii and Tribulus terrestris in the ratio about 1:1.

17. A process as claimed in claim 14, wherein the anti-carcinogenic activity of the said extract is confirmed by in-vivo experiments.

18. Use of the said extract as claimed in claim 14, wherein in the treatment of prostate cancer.
